(19) United States
(12) Patent Application Publication
(10) Pub. No.: US 2014/0066758 A1
(43) Pub. Date: Mar. 6, 2014

(54) SPINAL IMPLANT SYSTEM AND METHODS OF USE

(71) Applicants: Gregory Marik, Collierville, TN (US); Trevor Seck, Memphis, TN (US)

(72) Inventors: Gregory Marik, Collierville, TN (US); Trevor Seck, Memphis, TN (US)

(73) Assignee: WARSAW ORTHOPEDIC, INC., Memphis, TN (US)

(21) Appl. No.: 13/597,015

(22) Filed: Aug. 28, 2012

(51) Int. Cl. A61B 6/00; A61B 17/70

(52) U.S. Cl. 600/431

(57) ABSTRACT

A method for treating a spine disorder includes providing a bone fastener including a distal portion and a proximal portion including an axial opening and defining a longitudinal passageway. At least one lateral opening is disposed in communication with the longitudinal passageway. The axial opening is disposed in communication with the longitudinal passageway. The distal portion is engaged with a first vertebral surface. The second vertebral surface is spaced apart from the first vertebral surface. A radiopaque biologic is introduced through the axial opening and into the longitudinal passageway and delivered to a surgical site adjacent the first vertebral surface and the second vertebral surface. Systems for treating a spine disorder are disclosed.

### TECHNICAL FIELD

The present disclosure generally relates to medical devices for the treatment of spinal disorders, and more particularly to a spinal implant system and method including a biologic configured to be delivered to a surgical site.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Fusion and fixation treatments may employ implants with vertebrae to achieve arthrodesis. This disclosure describes an improvement over these prior art technologies.

### SUMMARY

In one embodiment, in accordance with the principles of the present disclosure, a method for treating a spine disorder is provided. The method includes the steps of: providing a bone fastener including a distal portion and a proximal portion, the proximal portion including an axial opening and defining a longitudinal passageway, the bone fastener defining at least one lateral opening disposed in communication with the longitudinal passageway, the axial opening being disposed in communication with the longitudinal passageway, the distal portion including an outer surface configured for penetrating tissue; engaging the distal portion with a first vertebral surface and a second vertebral surface of vertebrae, the second vertebral surface being spaced apart from the first vertebral surface; introducing a radiopaque biologic through the axial opening and into the longitudinal passageway; and delivering the radiopaque biologic to a surgical site adjacent the first vertebral surface and the second vertebral surface.

In one embodiment, in accordance with the principles of the present disclosure, a spinal implant system is provided. The spinal implant system includes a shaft having an outer surface configured for penetrating tissue. A head includes an axial opening and defines a longitudinal passageway. The axial opening is disposed in communication with the longitudinal passageway. At least one lateral opening is disposed in communication with the longitudinal passageway. A flowable radiopaque biologic is introduced through the axial opening into the longitudinal passageway and through the at least one lateral opening for delivery to a surgical site.

In one embodiment, the method for treating a spine disorder includes the steps of: providing a bone fastener extending along a longitudinal axis and including a distal portion, a proximal portion and an intermediate portion disposed between the proximal portion and the distal portion, the bone fastener defining a pair of lateral openings oriented to face opposing directions, the proximal portion including a first outer threaded surface, an axial opening and defining a longitudinal passageway disposed in communication with the axial opening and the lateral openings, the distal portion including a second outer threaded surface spaced apart from the first outer threaded surface and configured for penetrating tissue, the intermediate portion including an even outer surface and the lateral openings extending through the outer surface, the at least one lateral opening having an oblong shape and being disposed in parallel relation with the longitudinal axis; engaging the distal portion with a first vertebral surface and a second vertebral surface, the second vertebral surface being spaced apart from the first vertebral surface; introducing a flowable radiopaque biologic through the axial opening and into the longitudinal passageway, the biologic comprising barium, calcium phosphate, bismuth, iodine, tantalum, tungsten, metal beads, metal particles, or combinations thereof; and delivering the radiopaque biologic through the lateral openings to a surgical site adjacent the first vertebral surface and the second vertebral surface.

### BRIEF DESCRIPTION OF THE DRAWINGS

The present disclosure will become more readily apparent from the specific description accompanied by the following drawings, in which:

FIG. 1 is a perspective view of one particular embodiment of a component of a spinal implant system in accordance with the principles of the present disclosure;

FIG. 2 is a perspective view of the component shown in FIG. 1;

FIG. 3 is a side view of the component shown in FIG. 1;

FIG. 4 is a side cross sectional view of the component shown in FIG. 1;

FIG. 5 is a perspective view of one embodiment of a system in accordance with the principles of the present disclosure disposed with vertebrae;

FIG. 6 is a perspective view, in part phantom, of the system shown in FIG. 5 disposed with vertebrae;

FIG. 7 is a perspective view of one embodiment of a system in accordance with the principles of the present disclosure disposed with vertebrae;

FIG. 8 is a perspective view of the vertebrae shown in FIG. 7;

FIG. 9 is a perspective view of one particular embodiment of a component of a system in accordance with the principles of the present disclosure;

FIG. 10 is a perspective view of the component shown in FIG. 9;

FIG. 11 is a perspective view of one particular embodiment of a component of a system in accordance with the principles of the present disclosure;

FIG. 12 is a perspective view of the component shown in FIG. 11;

FIG. 13 is a perspective view of one particular embodiment of a component of a system in accordance with the principles of the present disclosure;

FIG. 14 is a perspective view of the component shown in FIG. 13; and

FIG. 15 is a perspective view of the component shown in FIG. 13.

Like reference numerals indicate similar parts throughout the figures.

### DETAILED DESCRIPTION

The exemplary embodiments of a surgical system, a bone fastener and related methods of use disclosed are discussed in terms of medical devices for the treatment of musculoskeletal disorders and more particularly, in terms of a spinal implant system including a bone fastener and a method for treating a vertebral column. In one embodiment, the bone fastener includes lateral ports or slots. In one embodiment, a flowable radiopaque biologic is injected through an instrument and into the bone fastener such that the flowable radiopaque biologic flows out of the bone fastener through the lateral ports or slots and into a surgical site or a location adjacent a surgical site. In one embodiment, the bone fastener is implanted into a facet joint such that the radiopaque biologic is delivered proximate to a superior articular facet and/or a inferior articular facet to promote fusion therebetween. In one embodiment, the spinal implant system includes a biologic including a radiopaque dye added to the biologic to enhance visualization of an anatomical area adjacent to an area in which the biologic is injected and/or the biologic itself. It is envisioned that the radiopaque dye may be configured to enhance medical imaging.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed system and methods of use may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of use of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

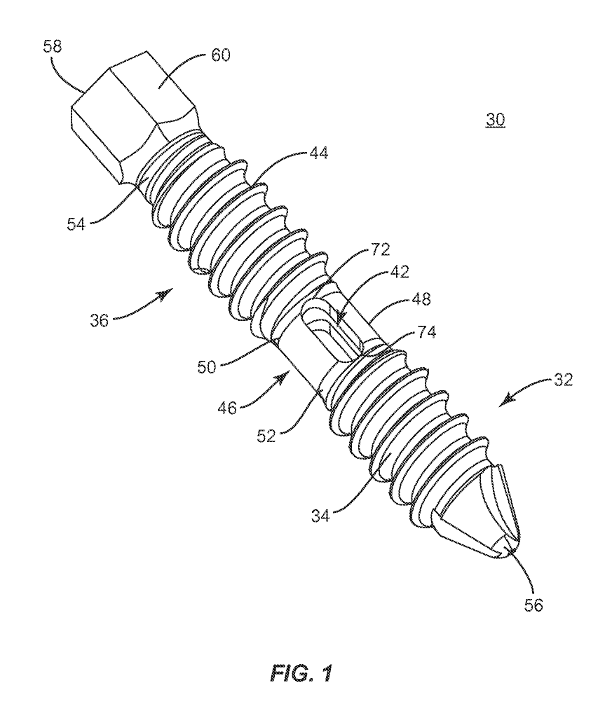

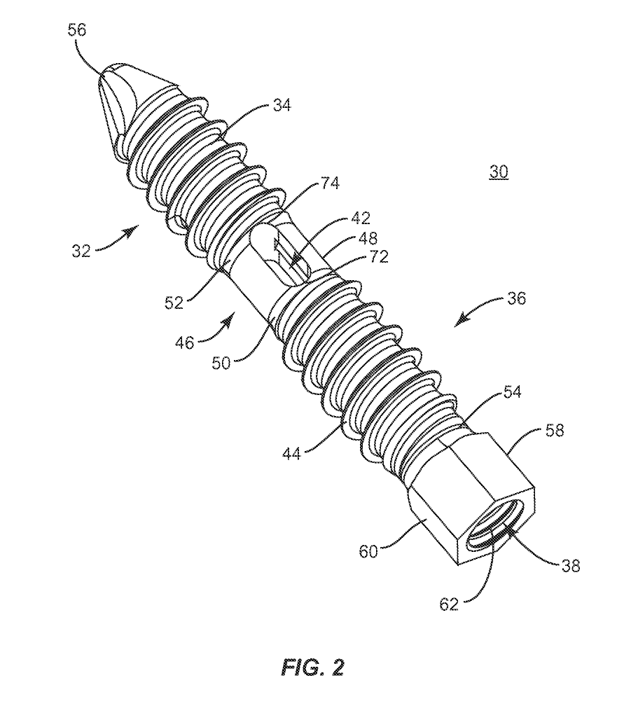

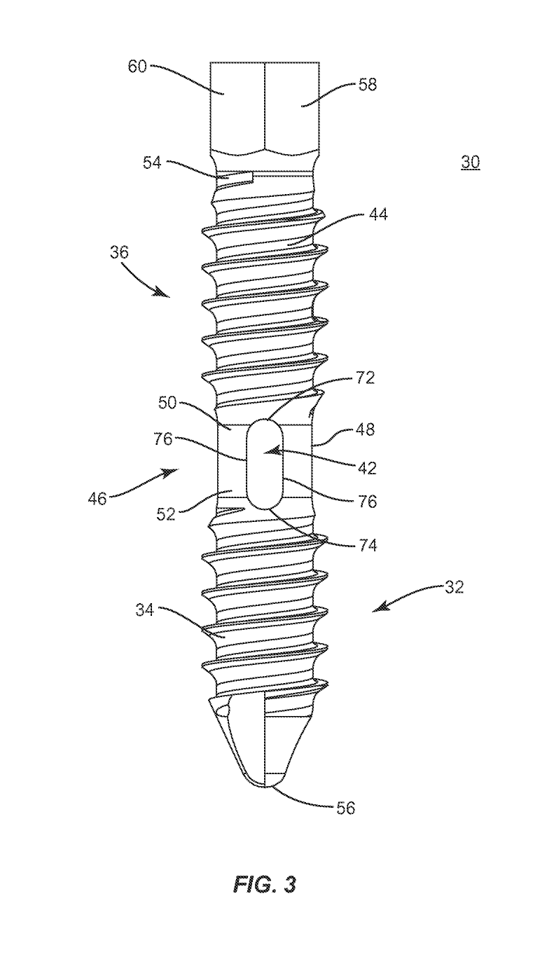

The following discussion includes a description of a surgical system including a bone fastener, related components and methods of employing the surgical system including the bone fastener in accordance with the principles of the present disclosure. Alternate embodiments are also disclosed. Reference will now be made in detail to the exemplary embodiments of the present disclosure, which are illustrated in the accompanying figures. Turning now to FIGS. 1-4, there is illustrated components of a surgical system including a bone fastener 30 in accordance with the principles of the present disclosure.

The components of the surgical system can be fabricated from biologically acceptable materials suitable for medical applications, including metals, synthetic polymers, ceramics and bone material and/or their composites, depending on the particular application and/or preference of a medical practitioner. For example, the components of the surgical system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the surgical system may have material composites, including the above materials, to achieve various desired characteristics such as strength, rigidity, elasticity, compliance, biomechanical performance, durability and radiolucency or imaging preference. The components of the surgical system, individually or collectively, may also be fabricated from a heterogeneous material such as a combination of two or more of the above-described materials. The components of the surgical system may be monolithically formed, integrally connected or include fastening elements and/or instruments, as described herein.

Fastener 30 defines a longitudinal axis a1 and includes a distal portion, such as, for example, a shaft 32 including a threaded outer surface 34 configured for penetrating tissue. It is contemplated that the thread form on surface 34 may extend the entire length of shaft 32 or may extend only part of the length of shaft 32. It is further contemplated that shaft 32 may include one thread form configuration or a plurality of different thread form configurations. It is envisioned that the thread form configurations on surface 34 may include a single thread turn or a plurality of discrete threads. It is further envisioned that other engaging structures may be located along surface 34, in place of or in addition to the thread form configurations discussed above, such as, for example, a nail configuration, barbs, expanding elements, raised elements and/or spikes to facilitate engagement with tissue, such as, for example, bone.

Fastener 30 includes a proximal portion 36 including an axial opening 38 extending through a proximal face of portion 36 and defining a longitudinal passageway 40 extending parallel to axis a1. Opening 38 is disposed in communication with passageway 40. Opening 38 has a substantially circular cross sectional configuration and passageway 40 has a substantially cylindrical cross sectional configuration. It is contemplated that all or only a portion of opening 38 and/or passageway 40 may be variously configured and dimensioned, such as, for example, circular, oval, oblong, triangular, square, rectangular, polygonal, irregular, uniform, non-uniform, offset, staggered, tapered, consistent or variable, depending on the requirements of a particular application. It is envisioned that passageway 40 may be disposed at alternate orientations relative to axis a1, such as, for example, transverse, perpendicular and/or other angular orientations such as acute or obtuse and/or may be offset or staggered and/or may be disposed at alternative angular orientations relative to axis a1, depending on the requirements of a particular application.

Passageway 40 has a width w that is less than a width w1 of opening 38. Fastener 30 includes a ledge 66 at an interface between opening 38 and passageway 40. Ledge 66 is disposed at an acute angle relative to axis a1 and is configured to guide a material, such as, for example, a fluid through opening 38 and into passageway 40. Ledge 66 is tapered and extends between an upper end 68 having a width equivalent to width w1 of opening 38 and a lower end having a width equivalent to width w of passageway 40.

Portion 36 includes a threaded outer surface 44 spaced apart from the threads on surface 34 such that the threads on surface 44 and the threads on surface 34 are non-continuous. It is contemplated that the thread form on surface 44 may extend the entire length of portion 36 or may extend only part of the length of portion 36. It is envisioned that the thread form configurations on surface 44 may include a single thread turn or a plurality of discrete threads. It is further envisioned that other engaging structures may be located along surface 44, in place of or in addition to the thread form configurations discussed above, such as, for example, a nail configuration, barbs, expanding elements, raised elements and/or spikes to facilitate engagement with tissue, such as, for example, bone.

In one embodiment, the thread form on surface 34 and/or the thread form on surface 44 has a major diameter that is 25% greater than a minor diameter of the thread form on surface 34 and/or the thread form on surface 44. It is envisioned that the thread form on surface 34 and/or the thread form on surface 44 may have a major diameter that is between about 15% and about 50% greater than a minor diameter of the thread form on surface 34 and/or the thread form on surface 44. In one embodiment, the thread form on surface 34 and/or the thread form on surface 44 has a pitch of 1.5, such that the distance from one thread to the next is 1.5 millimeters (mm). It is envisioned that the thread form on surface 34 and/or the thread form on surface 44 may have a pitch of 0.4, 0.45, 0.5, 0.7, 0.8, 1.0, 1.25, 1.5, 1.75, 2.0 or 2.5.

Fastener 30 includes an intermediate portion 46 positioned between portion 36 and shaft 32 having an even outer surface 48. It is envisioned that surface 48 may be threaded such that the thread form configurations on surface 34 and surface 44 are continuous. Portion 46 has a length L extending from a first end 50 to a second end 52. Portion 36 has a length L1 extending from a top portion 54 and end 50. Shaft 32 has a length L2 extending along axis a1 from end 52 to a tip 56. Length L1 and length L2 are approximately equivalent and length L is less than length L1 and length L2. In one embodiment, a start 64 of the thread form on surface 34 is disposed at a 25 degree angle relative to tip 56. It is envisioned that start 64 may be at an angle relative to tip 56 between about 10 degrees and about 45 degrees.

Portion 36 includes a head 58 proximal to portion 54 along axis a1 having an outer surface 60 configured to engage a tool, such as, for example, a driver to fix fastener 30 into tissue, such as, for example, bone. Head 58 has a hexagonal cross sectional configuration configured for engagement with a driver, such, for example, a hex socket tool. It is envisioned that head 58 may be variously configured and dimensioned, such as, for example, oval, oblong, square, rectangular, polygonal, irregular, uniform, non-uniform, offset, staggered, tapered, consistent or variable, depending on the requirements of a particular application. Opening 38 extends through head 58. An inner surface 62 of head 58 defines opening 38. In one embodiment, surface 62 is threaded to facilitate engagement of fastener 30 with an instrument, such as, for example, a fluid delivery device.

Fastener 30 includes at least one lateral opening 42 extending through portion 46 disposed in communication with passageway 40. In one embodiment, fastener 30 includes a pair of oblong openings 42 each extending parallel to axis a1. Openings 42 are oriented in opposing directions such that openings 42 are aligned with one another and define a transverse passageway extending through fastener 30. Openings 42 each extend from a first end 72 to a second end 74. Ends 72, 74 each have an arcuate shape and extend between planar side surfaces 76. It is envisioned that fastener 30 may include one or a plurality of openings 42. It is further envisioned that all or only a portion of openings 42 may have alternate cross section configurations, such as, for example, oval, oblong, triangular, square, polygonal, irregular, uniform, non-uniform, offset, staggered, undulating, arcuate, variable and/or tapered. It is contemplated that fastener 30 may have one or a plurality of openings 42 extending through portion 32 and/or portion 36 in addition to, or in place of, openings 42 extending through portion 46.

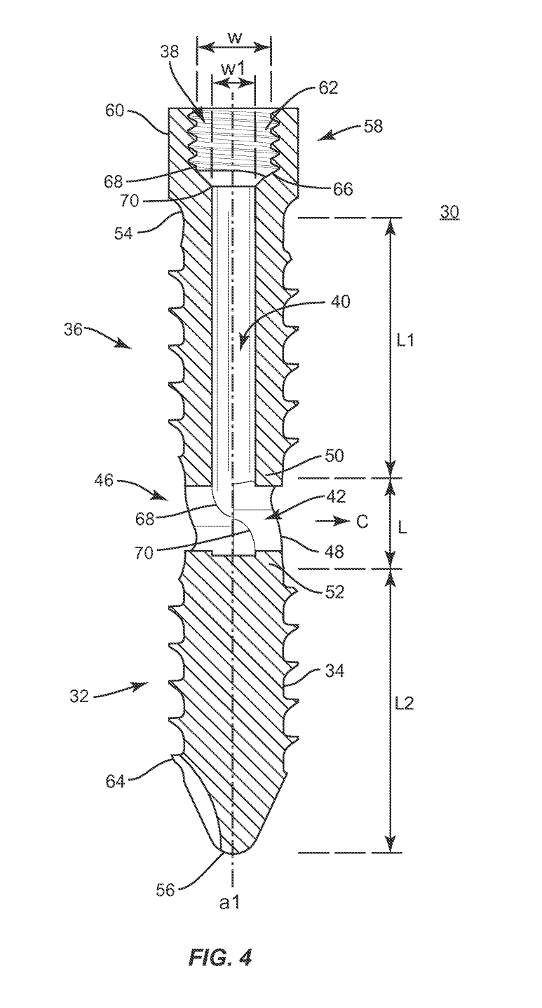

A distal end of passageway 40 includes a concave portion 68 continuous with a convex portion 70. Portions 68, 70 are positioned adjacent opening 42 to facilitate movement of a material disposed in passageway 40 through opening 42 such that the material moves out of opening 42 in a direction transverse to axis a1, such as, for example, the direction shown by arrow C in FIG. 4. It is envisioned that portions 68, 70 may be variously configured such that a material disposed in passageway 40 may move out of fastener 30 through opening 42 in alternate directions relative to axis a1, such as, for example, angular orientations such as acute or obtuse relative to axis a1, depending on the requirements of a particular application. It is envisioned that portions 68, 70 may be configured such that a material disposed in passageway 40 moves out of fastener 30 through opening 42 at an angle between about +/−1 degree to about +/−44 degrees relative to axis a1, or any angle therebetween.

A biologic 78 is disposed with and/or injected into fastener 30 for delivery to a surgical site or a location adjacent a surgical site. Biologic 78 is injected into opening 38. Biologic 78 flows from opening 38 into passageway 40. Biologic 78 flows through passageway 40 and exits fastener 30 through opening 42 for delivery to a surgical site or a location adjacent a surgical site. Biologic 78 is flowable such that biologic 78 has a consistency ranging from shape sustaining to readily deformable. It is envisioned that biologic 78 may be in the form of a suspension, solution, gel, cake, paste, putty, cream or filler.

Biologic 78 includes a component, such as, for example, a radiopaque dye, possessing the property of enhancing or intensifying X-ray contrast (radiopacity), relative to a formulation or composition lacking this component. The radiopaque dye improves the capability of visualizing biologic 78 and/or a surgical site adjacent a location in which biologic 78 is injected, which are often not directly observable, and thus allows a medical practitioner to evaluate the progress and/or result of a treatment, such as, for example, a spinal fusion, in an X-ray image. In one embodiment, biologic 78 includes barium, calcium phosphate, bismuth, iodine, tantalum, tungsten, metal beads, metal particles, or any combination thereof.

For the enhancement of X-ray contrast (radiopacity), all elements can be used for which the atomic number in the periodic table of elements is greater than 20 and thereby higher than that of calcium. Biologic 78 may include substances for enhancing radiopacity including metals, inorganic metal compounds such as metal oxides, metal nitrides, metal carbides, metal silicids, metal halides, metal phosphates, metal gluconates, metal citrates, metal fumarates and metal sulfates, metal-organic compounds based on iron, titan, tantalum, gold, silver, rare earth elements, yttrium, ytterbium, molybdenum, zirconium, niobium, ruthenium, rhodium, palladium and tungsten, iron, rare earth elements (lanthanides), cerium, gadolinium, iron phosphate, iron oxide, iron hydroxide or iron-compound with organic acids like iron citrate, tungsten salicylate, water soluble lanthanum or rare earth compounds such as lanthanum acetate, lanthanum nitrate, lanthanum sulfate, lanthanum ammonium nitrate, cerium citrate, cerium nitrate, cerium chloride, cerium ammonium sulfate, gadolinium compounds including gadolinium fluorid, gadolinium chlorid, gadolinium chelates including gadolinium diethylenetriamino pentaacetate, Gadoteridol, barium, calcium phosphate, bismuth, iodine, tungsten or any combination thereof.

In one embodiment, metals and inorganic metal compounds such as oxides, nitrides, carbides, silicides and halides that enhance X-ray contrast may be included in biologic 78 in a fine particulate form. In one embodiment, the average particle size of the particulate metal or inorganic metal compound lies in the range of about 0.1 nm to about 10.0 μm to permit flowability. It is envisioned that the average particle size of the particulate metal or inorganic metal compound may lie in the range of about 5.0 nm to about 1.0 μm; about 5.0 nm to about 500 nm; or about 5.0 nm to about 100 nm.

In one embodiment, sintered materials, including highly sintered materials, may be used as the radiopacifier additive(s). Sintered materials include sintered hydroxyapatite and sintered tricalcium phosphate, for example. In one embodiment, the sintered material is in a fine particulate form.

In one embodiment, biologic 78 includes at least one bone growth material to promote bone growth in a surgical site or a location adjacent a surgical site. The bone growth material may include solid materials, such as, for example, bone graft, allograft chips, autogenous morselized bone graft, strips of autogenous bone graft, demineralized bone matrix in powder, paste, putty, gel, strip, or other forms, xenografts and fired bone. The solids can also be bone graft substitutes, such as hydroxyapatite, calcium carbonate, beta tricalcium phosphate, calcium sulfate or mineralized collagen, natural or synthetic polymers such as collagen particles, meshes, sponges, and gels, hyaluronic acid and derivatives thereof, liposomes or other natural biomaterials known as potential implants, or carriers of therapeutic agents, such as cytokines, growth factors, cells, antibiotics, analgesics, chemotherapeutic drugs, and the like. The bone growth material may include synthetic polymers, such as, for example, alpha-hydroxy polyesters, including polylactic acid, polyglycolic acid and their copolymers, polydioxanone, as well as poly methyl methacrylate, separately, in mixture or in admixture with any of the therapeutic agents described.

The bone growth material(s) used may include a therapeutically effective amount of a growth factor including, for example, osteoinductive agents (e.g. agents that cause new bone growth in an area where there was none before) and/or osteoconductive agents (e.g. agents that cause ingrowth of cells into and/or through the matrix). Osteoinductive agents can be polypeptides or polynucleotides compositions. Polynucleotide compositions of the osteoinductive agents include, but are not limited to, isolated Bone Morphogenic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta) polynucleotides. Polynucleotide compositions of the osteoinductive agents include, but are not limited to, gene therapy vectors harboring polynucleotides encoding the osteoinductive polypeptide of interest. Gene therapy methods often utilize a polynucleotide, which codes for the osteoinductive polypeptide operatively linked or associated to a promoter or any other genetic elements necessary for the expression of the osteoinductive polypeptide by the target tissue. Suitable gene therapy vectors include, but are not limited to, gene therapy vectors that do not integrate into the host genome. Other suitable gene therapy vectors include, but are not limited to, gene therapy vectors that integrate into the host genome.

In one embodiment, fastener 30 is treated with at least one of the bone growth materials discussed above to promote bone ingrowth between fastener 30 and tissue into which fastener 30 is fixed, such as, for example, a first vertebral surface and/or a second vertebral surface. In one embodiment, the first vertebral surface is a superior articular facet of a vertebra and the second vertebral surface is an inferior articular facet of an adjacent vertebra. It is envisioned that the bone growth material(s) can be applied to the exterior of fastener 30, such as, for example, on surfaces 34, 44 and/or 48. The bone growth material may be applied to the exterior of fastener 30 by electrospraying, ionization spraying or impregnating, vibratory dispersion (including sonication), nozzle spraying, compressed-air-assisted spraying, brushing and/or pouring.

Biologic 78 may include one or more therapeutic agent in addition to, or in place of, the bone growth materials discussed above. Therapeutic agents include but are not limited to IL-1 inhibitors, such Kineret® (anakinra), which is a recombinant, non-glycosylated form of the human interleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogues (which reduce TNF-α production by macrophages), quinapril (an inhibitor of angiotensin II, which upregulates TNF-α), interferons such as IL-11 (which modulate TNF-α receptor expression), and aurin-tricarboxylic acid (which inhibits TNF-α may also be useful as therapeutic agents for reducing inflammation. It is further contemplated that where desirable a pegylated form of the above may be used. Examples of still other therapeutic agents include NF kappa B inhibitors such as antioxidants, such as dilhiocarbamate, and other compounds, such as, for example, sulfasalazine.

Examples of therapeutic agents suitable for use also include, but are not limited to, an anti-inflammatory agent, analgesic agent, or osteoinductive growth factor or a combination thereof. Anti-inflammatory agents include, but are not limited to, apazone, celecoxib, diclofenac, diflunisal, enolic acids (piroxicam, meloxicam), etodolac, fenamates (mefenamic acid, meclofenamic acid), gold, ibuprofen, indomethacin, ketoprofen, ketorolac, nabumetone, naproxen, nimesulide, salicylates, sulfasalazine[2-hydroxy-5-[4-[C2-pyridinylamino)sulfonyl]azo]benzoic acid, sulindac, tepoxalin, and tolmetin; as well as antioxidants, such as dithiocarbamate, steroids, such as cortisol, cortisone, hydrocortisone, fludrocortisone, prednisone, prednisolone, methylprednisolone, triamcinolone, betamethasone, dexamethasone, beclomethasone, fluticasone or a combination thereof.

Suitable analgesic agents include, but are not limited to, acetaminophen, bupivicaine, fluocinolone, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papavereturn, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof. In some embodiments, a statin may be used. Statins include, but is not limited to, atorvastatin, simvastatin, pravastatin, cerivastatin, mevastatin, velostatin, fluvastatin, lovastatin, rosuvastatin and fluindostatin (Sandoz XU-62-320), dalvastain, eptastatin, pitavastatin, or pharmaceutically acceptable salts thereof or a combination thereof. In one embodiment, the statin includes mixtures of (+)R and (−)-S enantiomers of the statin. In one embodiment, the statin includes a 1:1 racemic mixture of the statin.

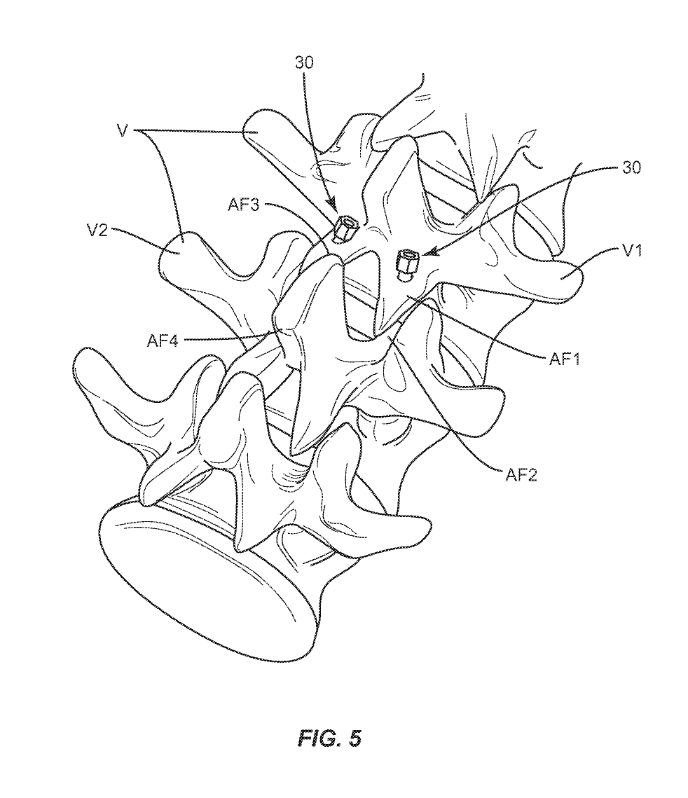

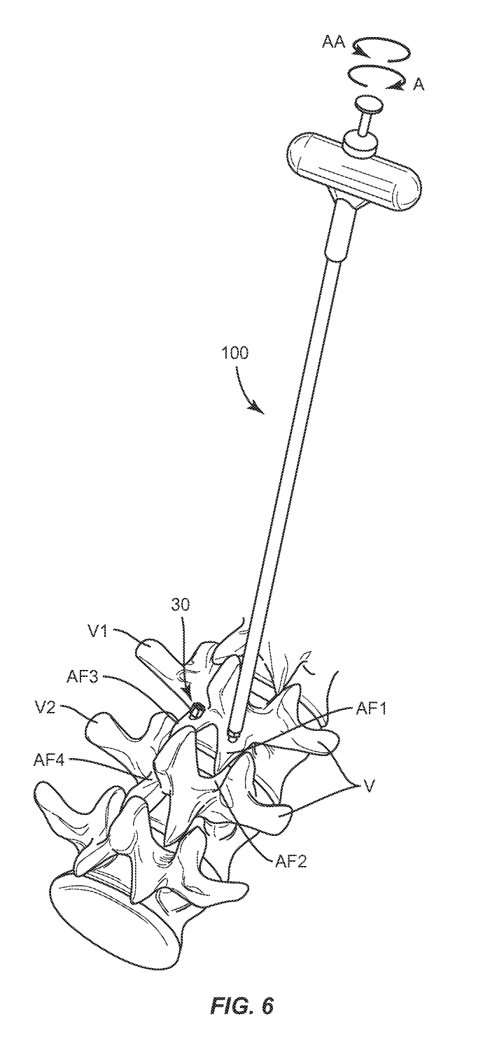

In assembly, operation and use, a surgical system including fastener 30, similar to that described above, is employed with a surgical procedure for treatment of a spinal disorder affecting a section of a spine of a patient, as discussed herein. In particular, the surgical system, including fastener 30, is employed with a surgical procedure for treatment of a condition or injury of an affected section of the spine including vertebrae V, as shown in FIGS. 5-8. In one embodiment, the system including fastener 30 is attached to vertebrae V for a surgical arthrodesis procedure, such as a fusion of the affected section of the spine, such as, for example, a facet joint of vertebrae V, to facilitate healing and therapeutic treatment. In one embodiment, the system including fastener 30 is attached to vertebrae V for a posterolateral fusion procedure.

In one embodiment, a surgeon employs a minimally invasive technique and makes an incision in the skin of a patient over and in substantial alignment with a surgical site, which includes vertebrae V. The surgical system includes a dilator (not shown) employed to separate the muscles and tissues to create a passageway along a desired trajectory to the surgical site through which the surgery may be performed. It is contemplated that the dilator may include one or a plurality of dilators, and/or employ a retractor, to gradually separate muscle and tissue to create a portal including the passageway. It is further contemplated that the dilator may be configured as an in-situ guidance instrument and may include an endoscope camera tip. The system includes a retractor (not shown) positioned and docked adjacent the surgical site over the incision.

It is envisioned that the components of the surgical system may be used in any existing surgical method or technique including open surgery, mini-open surgery, minimally invasive surgery including percutaneous surgical implantation, whereby vertebrae V are accessed through a micro-incision, or sleeve that provides a protected passageway to the area. Once access to the surgical site is obtained, the particular surgical procedure is performed for treating the spinal disorder. The components of the surgical system, including fastener 30 are employed to augment the surgical treatment. The surgical system may be may be completely or partially revised, removed or replaced.

The surgeon makes a small incision in the skin of a patient over and in substantial alignment with the surgical site, which includes vertebrae V. A dilator (not shown) may be used that includes a cannula, mini-open retractor or tube, which creates and defines at least one passageway for passage of the components of the system, discussed herein, to the surgical site from a desired trajectory.

A preparation instrument(s) (not shown) is inserted within the at least one passageway and disposed adjacent superior articular facets AF1, AF3 of a vertebra V1 and inferior articular facets AF2, AF4 of a vertebra V2 adjacent vertebra V1. For example, a preparation instrument, such as a Cobb elevator, a surgical drill and/or a sleeved burr is disposed in the passageway and adjacent the surgical site. The preparation instrument, with the assistance of image guidance, prepares articular facets AF1-AF4. It is envisioned that the preparation instrument(s) may include rasps, curettes and/or a rotating tissue remover such as a rapid disc removal system that can be low profile to cut and remove disc and/or bone material simultaneously. The preparation instrument(s) is employed to remove tissue and fluids adjacent tissues and/or bone, scrape and/or remove tissue from vertebral surfaces, as well as aspirate and irrigate the region according to the requirements of a particular surgical application. The preparation instrument is removed from the passageway thereafter.

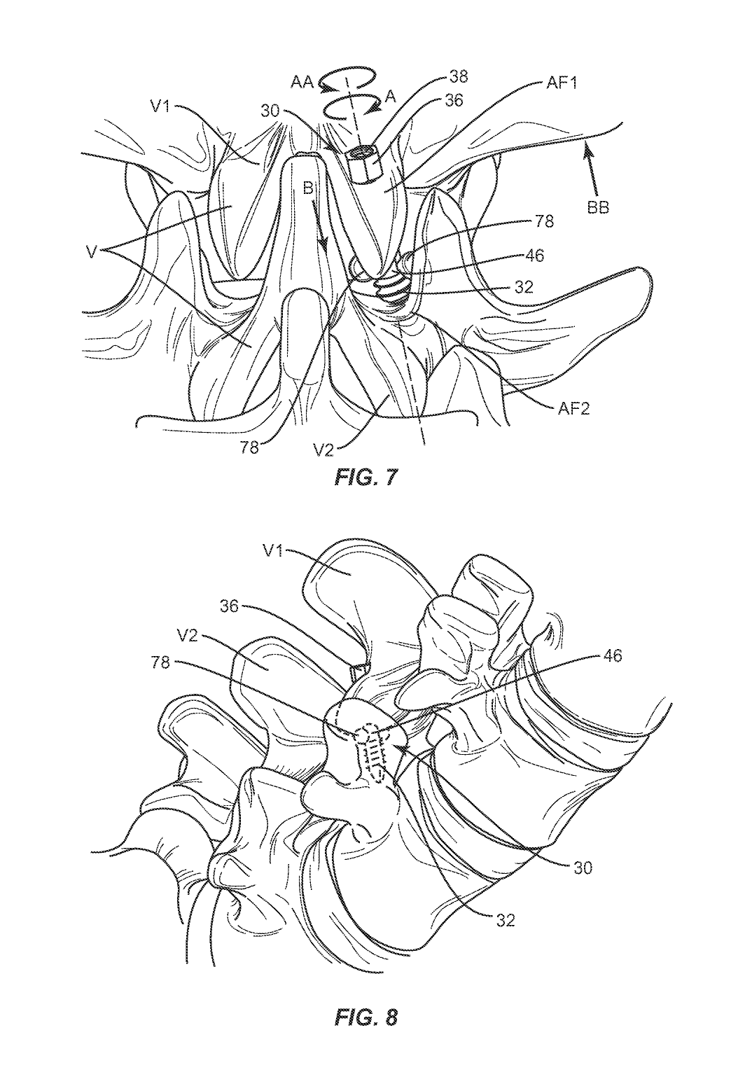

Pilot holes are made in articular facets AF1-AF4 for receiving fasteners 30, as shown in FIG. 5. A first fastener 30 is inserted or otherwise connected to superior articular facet AF1 such that tip 56 is positioned within the pilot hole in superior articular facet AF1 and the threads on surface 34 engage tissue. A driver 100 is engaged with head 58 to rotate first fastener 30 in a first direction, such as, for example, the direction shown by arrow A such that first fastener 30 translates within superior articular facet AF1 in the direction shown by arrow B, in FIG. 7.

First fastener 30 is rotated until tip 56 is positioned within the pilot hole in inferior articular facet AF2 and the threads on surface 34 engage tissue. Portion 36 is fixed within superior articular facet AF1, portion 32 is fixed within inferior articular facet AF2 and portion 46 is positioned between superior AF1 and inferior articular facet AF2. Fastener 30 may be rotated in the direction shown by arrow A or in the direction shown by arrow AA such that first fastener 30 translates, in the direction shown by arrow BB in FIG. 7, to adjust the position of portion 46 and/or openings 42 relative to superior articular facet AF1 and/or inferior articular facet AF2. A second fastener 30 is similarly disposed for fixation with superior articular facet AF3 and inferior articular facet AF4.

Biologic 78 is injected into opening 38 of each fastener 30. Biologic 78 flows from opening 38 into passageway 40. Biologic 78 flows through passageway 40 and exits fastener 30 through openings 42 for delivery to a location between the superior articular facets and the inferior articular facets. In one embodiment, a pressurized gas is injected into opening 38 and/or passageway 40 to facilitate flow of biologic 78 from opening 38 into passageway 40 and/or from passageway 40 through openings 42. In one embodiment, biologic 78 is introduced into opening 38 and/or passageway 40 under pressure and/or gravity.

In one embodiment, an instrument, such as, for example, a cannula having a threaded outer surface is threaded into surface 62 such that the threads on the outer surface of the cannula engage the threads on surface 62 to engage the cannula with fastener 30. Biologic 78 may be introduced through the cannula directly into passageway 40.

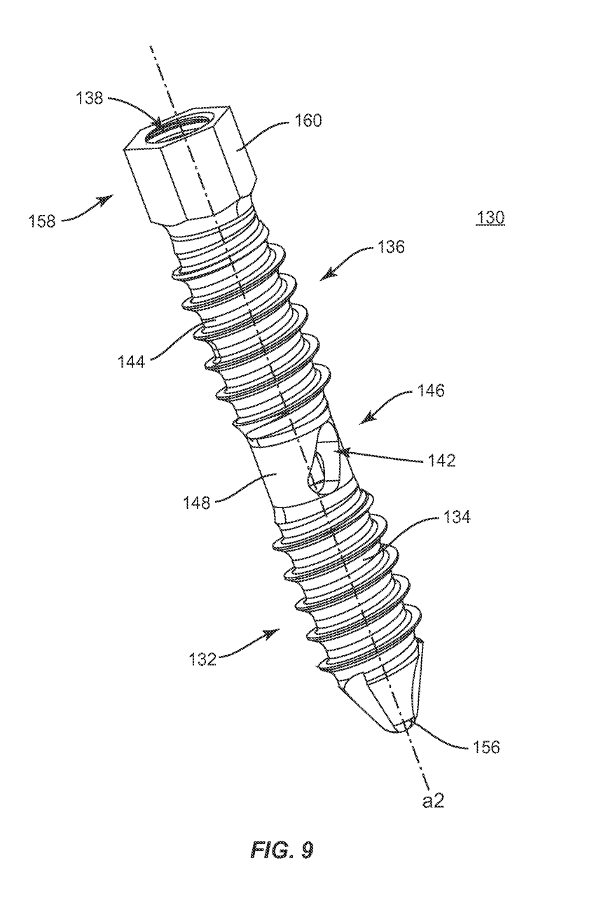

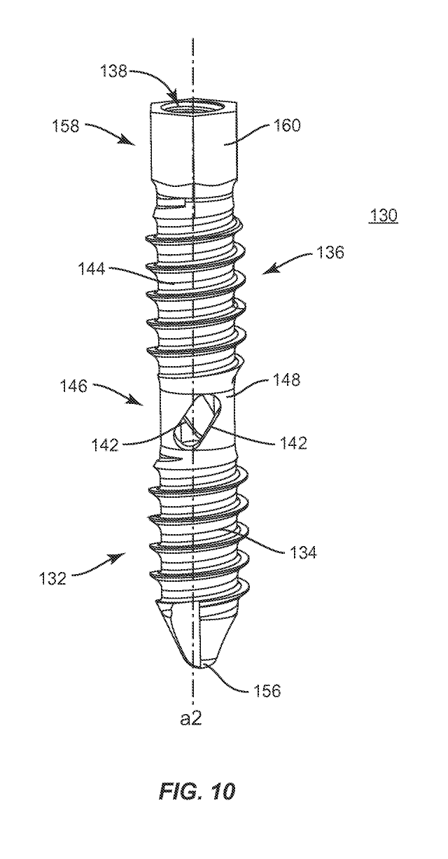

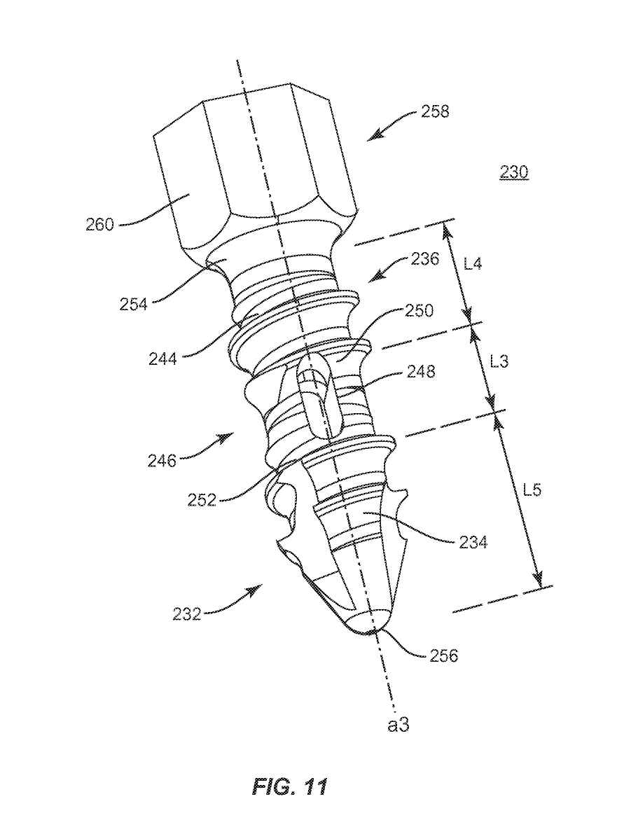

In one embodiment, shown in FIGS. 9 and 10, the surgical system includes a fastener 130, similar to fastener 30 and related methods described. Fastener 130 defines a longitudinal axis a2 and includes a shaft 132 including a threaded outer surface 134 configured for penetrating tissue.

Fastener 130 includes a proximal portion 136 including an axial opening 138 and defining a longitudinal passageway (not shown) having a configuration similar to that of passageway 40 extending parallel to axis a2. Opening 138 is disposed in communication with the passageway. Opening 138 has a substantially circular cross sectional configuration and the passageway has a substantially cylindrical cross sectional configuration. Portion 136 includes a threaded outer surface 144 spaced apart from surface 134. Portion 136 includes a head 158 having an outer surface 160 configured to engage a tool, such as, for example, a driver to fix fastener 130 into tissue, such as, for example, bone. Head 158 has a hexagonal cross sectional configuration configured for engagement with a driver, such, for example, a hex socket tool.

Fastener 130 includes an intermediate portion 146 positioned between portion 136 and shaft 132 having an even outer surface 148. Fastener 130 includes a pair of oblong openings 142 disposed in communication with the passageway and oriented in opposing directions. Each of openings 142 are oriented at an acute angle relative to axis a2. Openings 142 are each oriented at the same angle relative to axis a2 on opposite sides of fastener 130 such that openings 142 overlap, as shown in FIG. 10. It is envisioned that openings 142 may be oriented at different angles relative to axis a2. It is further envisioned that openings 142 may be oriented such that openings 142 are aligned with one another.

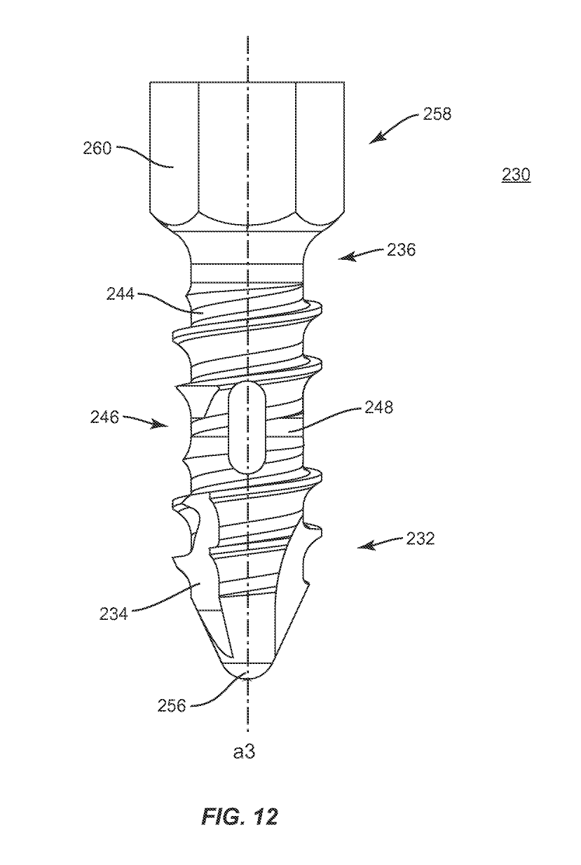

In one embodiment, shown in FIGS. 11 and 12, the surgical system includes a fastener 230, similar to fastener 30 and related methods described. Fastener 230 defines a longitudinal axis a3 and has a shaft 232 including a threaded outer surface 234 configured for penetrating tissue.

Fastener 230 includes a proximal portion 236 including an axial opening (not shown) having a configuration similar to that of openings 38, 138 and defining a longitudinal passageway (not shown) having a configuration similar to that of passageway 40 extending parallel to axis a3. The axial opening is disposed in communication with the passageway and has a substantially circular cross sectional configuration. The passageway has a substantially cylindrical cross sectional configuration. Portion 236 includes a threaded outer surface 244 spaced apart from surface 234. Portion 236 includes a head 258 proximal to portion 254 along axis a3 having an outer surface 260 configured to engage a tool, such as, for example, a driver to fix fastener 230 into tissue, such as, for example, bone. Head 258 includes six planar side surfaces such that head 258 has a hexagonal cross sectional configuration configured for engagement with a driver, such, for example, a hex socket.

Fastener 230 includes an intermediate portion 246 positioned between portion 236 and shaft 232 having a smooth or even outer surface 248 that is free of threads such that the thread form configurations on surface 234 and surface 244 are non-continuous. It is envisioned that surface 248 may be threaded such that the thread form configurations on surface 234 and surface 244 are continuous. Portion 246 has a length L3 extending from a first end 250 to a second end 252. Portion 236 has a length L4 extending from a top portion 254 and end 250. Shaft 232 has a length L5 extending along axis a3 from end 252 to a tip 256. Length L3 and length L4 are approximately equivalent and length L5 is greater than length L3 and length L4. It is envisioned that lengths L3, L4 and L5 may all be equivalent. It is further envisioned that length L3 may be greater than length L4 and/or length L5. Head 258 has a width that is greater than length L3, length L4 and/or length L5. It is envisioned that head 258 may have a width that is less than at least one of length L3, length L4 and length L5.

Fastener 230 includes at least one lateral opening 242 extending through portion 246 disposed in communication with the passageway. Fastener 230 includes a pair of oblong openings 242 each extending parallel to axis a3. Openings 242 are oriented in opposing directions such that openings 242 are aligned with one another and define a transverse passageway extending through fastener 230.

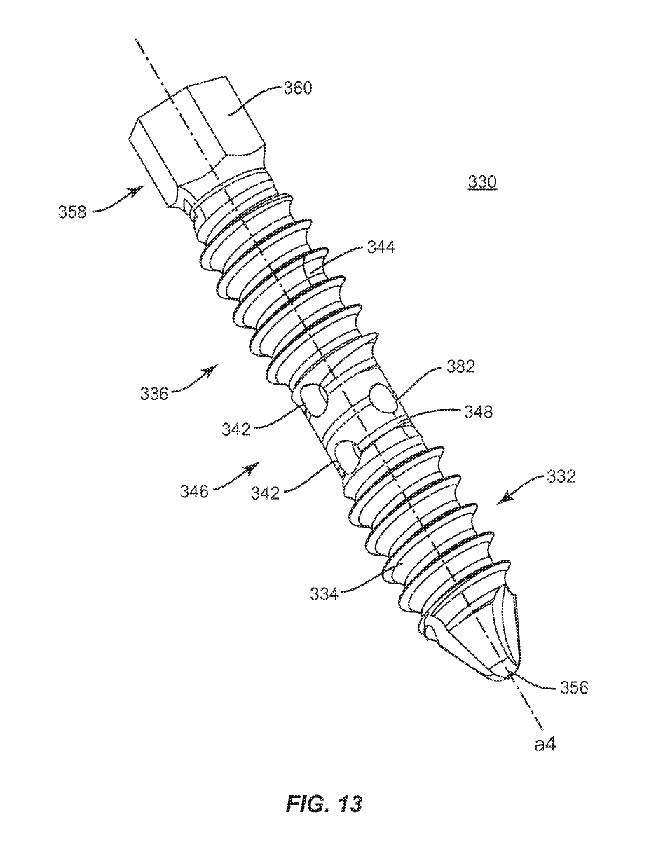

In one embodiment, shown in FIGS. 13-15, the surgical system includes a fastener 330, similar to fastener 30. Fastener 330 defines a longitudinal axis a4 and has a shaft 332 including a threaded outer surface 334 configured for penetrating tissue.

Fastener 330 includes a proximal portion 336 including an axial opening (not shown) having a configuration similar to that of openings 38, 138 and defining a longitudinal passageway (not shown) having a configuration similar to that of passageway 40 extending parallel to axis a4. The opening is disposed in communication with the passageway. The opening has a substantially circular cross sectional configuration and the passageway has a substantially cylindrical cross sectional configuration. Portion 336 includes a threaded outer surface 344 spaced apart from surface 334. Portion 336 includes a head 358 having an outer surface 360 configured to engage a tool, such as, for example, a driver to fix fastener 330 into tissue, such as, for example, bone. Head 358 has a hexagonal cross sectional configuration configured for engagement with a driver, such, for example, a hex socket tool.

Fastener 330 includes an intermediate portion 346 positioned between portion 336 and shaft 332 having an even outer surface 348. Fastener 330 includes a plurality of lateral openings 342, 362, 382 extending through portion 346 disposed in communication with the passageway. In one embodiment, fastener 330 includes a first pair of spaced apart circular openings 342 each extending parallel to axis a4 such that the first pair of openings 342 are coaxial on one side of fastener 330 and a second pair of spaced apart circular openings 362 each extending parallel to axis a4 such that the second pair of openings 362 are coaxial on an opposite side of fastener 330 such that openings 342 and openings 362 face opposing directions.

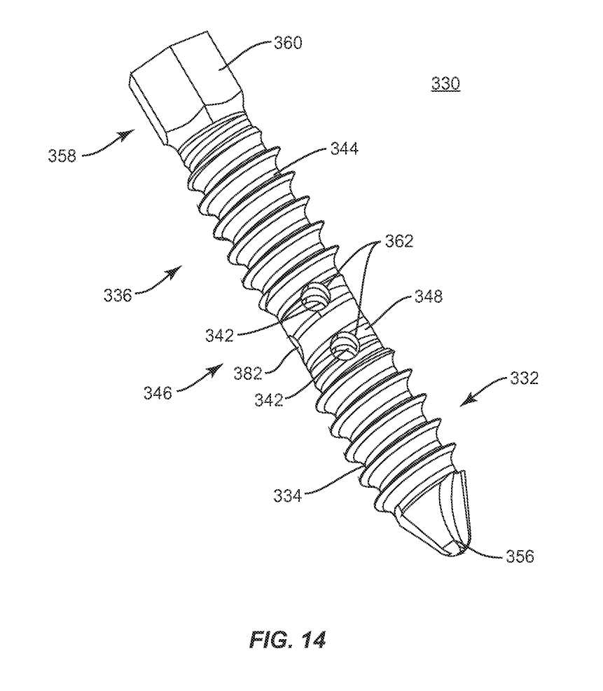

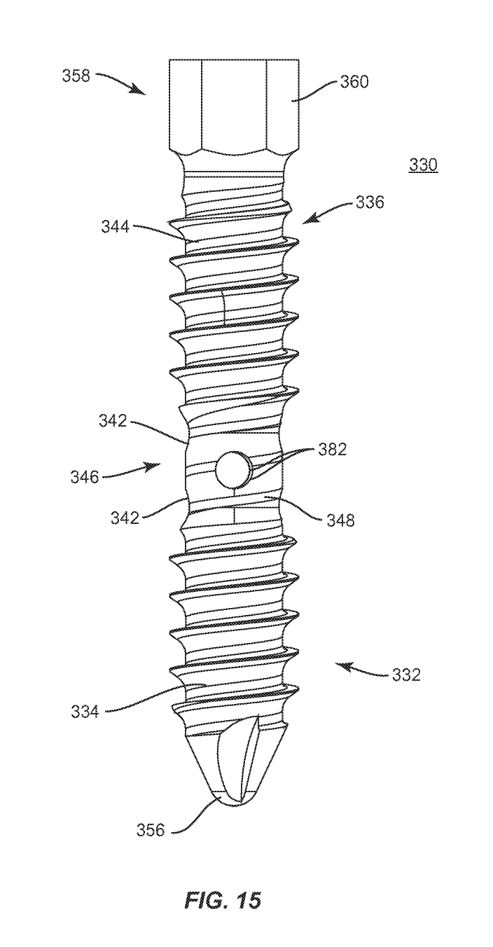

Openings 342 are aligned with openings 362 such that openings 342 and openings 362 define two transverse passageways extending through fastener 330, as shown in FIG. 14. Fastener 330 includes a third pair of circular openings 382 each extending parallel to axis a4. One of openings 382 is positioned between openings 342 and openings 362 on one side of fastener 330 and another opening 382 is positioned between openings 342 and openings 362 on an opposite side of fastener 330. Each of openings 382 are positioned equidistant from openings 342 and openings 362. Openings 382 are oriented in opposing directions such that openings 382 are aligned with one another and define a transverse passageway extending through fastener 330, as shown in FIG. 15.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for treating a spine disorder, the method comprising the steps of:
providing a bone fastener including a distal portion and a proximal portion, the proximal portion including an axial opening and defining a longitudinal passageway, the bone fastener defining at least one lateral opening disposed in communication with the longitudinal passageway, the axial opening being disposed in communication with the longitudinal passageway, the distal portion including an outer surface configured for penetrating tissue;
engaging the distal portion with a first vertebral surface and a second vertebral surface of vertebrae, the second vertebral surface being spaced apart from the first vertebral surface;
introducing a radiopaque biologic through the axial opening and into the longitudinal passageway; and
delivering the radiopaque biologic to a surgical site adjacent the first vertebral surface and the second vertebral surface.

2. A method as recited in claim 1, wherein the biologic comprises barium, calcium phosphate, bismuth, iodine, tantalum, tungsten, metal beads, metal particles, or combinations thereof.

3. A method as recited in claim 1, wherein the radiopaque biologic is flowable.

4. A method as recited in claim 1, wherein the radiopaque biologic comprises bone graft.

5. A method as recited in claim 1, wherein at least a portion of the bone fastener is treated with a bone growth promoting material to promote bone ingrowth between the bone fastener, the first vertebral surface and the second vertebral surface.

6. A method as recited in claim 1, wherein the first vertebral surface is a superior articular facet of a vertebra and the second vertebral surface is an inferior articular facet of an adjacent vertebra.

7. A method as recited in claim 1, wherein the proximal portion includes a first outer threaded surface and the distal portion includes a second outer threaded surface spaced apart from the first outer threaded surface, and the bone fastener includes an intermediate portion positioned between the proximal portion and the distal portion.

8. A method as recited in claim 1, wherein the proximal portion includes a first outer threaded surface and the distal portion includes a second outer threaded surface spaced apart from the first outer threaded surface, the bone fastener including an intermediate portion positioned between the proximal portion and the distal portion, the intermediate portion having an even outer surface and the at least one lateral opening extending through the outer surface.

9. A method as recited in claim 1, wherein the bone fastener extends along a longitudinal axis and the at least one lateral opening has an oblong shape and is disposed in parallel relation with the longitudinal axis.

10. A method as recited in claim 1, wherein the bone fastener extends along a longitudinal axis and includes a pair of lateral openings oriented to face opposing directions, the lateral openings each having an oblong shape and being disposed parallel with the longitudinal axis.

11. A method as recited in claim 1, wherein the bone fastener extends along a longitudinal axis and includes a pair of lateral openings oriented to face opposing directions, the lateral openings each having an oblong shape and being disposed at an acute angle relative to the longitudinal axis.

12. A method as recited in claim 1, wherein the bone fastener extends along a longitudinal axis and includes a pair of lateral openings opposite one another, the lateral openings each having an oblong shape, one of the lateral openings being disposed at a first acute angle relative to the longitudinal axis and the other lateral opening being disposed at a second acute angle relative to the longitudinal axis, the second acute angle being different from the first acute angle.

13. A method as recited in claim 1, wherein the bone fastener extends along a longitudinal axis and the at least one lateral opening includes a pair of spaced apart circular openings disposed along the longitudinal axis.

14. A method as recited in claim 1, wherein the at least one lateral opening includes a plurality of spaced apart circular openings, at least two of the lateral openings being coaxial.

15. A method as recited in claim 1, wherein the proximal portion includes an inner threaded surface defining the opening.

16. A method as recited in claim 1, wherein the proximal portion has a hexagonal configuration configured for engagement with a driving tool.

17. A method as recited in claim 1, wherein the proximal portion has a maximum diameter that is greater than a maximum diameter of the distal portion.

18. A spinal implant system comprising:
at least one bone fastener comprising
a shaft including an outer surface configured for penetrating tissue,
a head including an axial opening and defining a longitudinal passageway, the axial opening being disposed in communication with the longitudinal passageway, and
at least one lateral opening disposed in communication with the longitudinal passageway; and
a flowable radiopaque biologic;
wherein the radiopaque biologic is introduced through the axial opening into the longitudinal passageway and through the at least one lateral opening for delivery to a surgical site.

19. A method for treating a spine disorder, the method comprising the steps of:
providing a bone fastener extending along a longitudinal axis and including a distal portion, a proximal portion and an intermediate portion disposed between the proximal portion and the distal portion, the bone fastener defining a pair of lateral openings oriented to face opposing directions, the proximal portion including a first outer threaded surface, an axial opening and defining a longitudinal passageway disposed in communication with the axial opening and the lateral openings, the distal portion including a second outer threaded surface spaced apart from the first outer threaded surface and configured for penetrating tissue, the intermediate portion including an even outer surface and the lateral openings extending through the outer surface, the at least one lateral opening having an oblong shape and being disposed in parallel relation with the longitudinal axis;
engaging the distal portion with a first vertebral surface and a second vertebral surface, the second vertebral surface being spaced apart from the first vertebral surface;
introducing a flowable radiopaque biologic through the axial opening and into the longitudinal passageway, the biologic comprising barium, calcium phosphate, bismuth, iodine, tantalum, tungsten, metal beads, metal particles, or combinations thereof; and
delivering the radiopaque biologic through the lateral openings to a surgical site adjacent the first vertebral surface and the second vertebral surface.

20. A method as recited in claim 19, wherein the first vertebral portion is a superior articular facet of a vertebra and the second vertebral portion is an inferior articular facet of an adjacent vertebra.
